(19) United States
(12) Patent Application Publication
(10) Pub. No.: US 2024/0077220 A1
(43) Pub. Date: Mar. 7, 2024

(54) UV-C BASED ANTI-MICROBIAL AIR CIRCULATION SYSTEM

(71) Applicant: Worthington Armstrong Venture, Malvern, PA (US)

(72) Inventors: Joshua L. NEAL, Phoenixville, PA (US); Nicholas SHAFFER, Norristown, PA (US); Paloma N. Montero Lopez, Glenmoore, PA (US)

(73) Assignee: Worthington Armstrong Venture, Malvern, PA (US)

(21) Appl. No.: 18/239,147

(22) Filed: Aug. 29, 2023

Related U.S. Application Data
(60) Provisional application No. 63/404,217, filed on Sep. 7, 2022.

(51) Int. Cl. F24F 8/22; A61L 9/20; F24F 7/003

(52) U.S. Cl. CPC F24F 8/22; A61L 9/20; F24F 7/003; A61L 2209/15

(57) ABSTRACT

An UV-C based anti-microbial air circulation system for a suspended ceiling. The air circulation system comprises a UV light assembly having an internal chamber containing at least one UVC light. The UV light assembly is bookended by an intake assembly on one side and an exhaust assembly on the opposite side. An internal channel flows from an intake opening into the UV light assembly and out an exhaust opening. Angled fans move the air through the system so that air is exposed to at least one tenth of one second of UV-C light to disinfect it.

### RELATED APPLICATION

This application claims the benefit of priority to U.S. Provisional Patent Application Ser. No. 63/404,217, filed on Sep. 7, 2022, the contents of which are incorporated in this application by reference.

### FIELD OF THE INVENTION

The invention relates generally to an anti-microbial air circulation system. More particularly, the invention relates to an anti-microbial UV-C based air purification system that: is capable of fitting within a suspended ceiling grid, cycles environmental air through for cleaning, but prevents the UV-C light used to disinfect the air from escaping into the environment, however, the system is not required to be built into the ceiling structure.

### BACKGROUND

As the world continues to grapple with the reality of the coronavirus, global research communities are racing to develop practical solutions to adjust to the new challenges. One such challenge is the control of indoor air quality in the COVID-19 era and beyond. As the COVID-19 virus, with average aerodynamic diameter up to 80-120 nm, is viable as aerosol in indoor atmospheres for more than 3 hours, providing an air circulation system that kills this virus along with other microbes is desirable.

Anti-microbial modules to disinfect air have been proposed to be placed within the indoor environment's HVAC system. The problem with such proposals is the HVAC system typically has a limited number of air intakes and runs intermittently. As a result, air purification is only intermittent and the aerosolized virus can travel a significant distance between introduction to the environment—via a cough or sneeze—and when the air enters the HVAC system for treatment. During such travels, the virus can potentially infecting additional individuals in the environment. As a result, more area-specific treatment solutions are needed.

One proposal is to place ultraviolet (UV) lights, for example, within the suspended ceiling structure of office space. Suspended ceilings utilize a grid framework that includes main grid elements intersected by cross grid elements therebetween. The main and cross elements form a grid of polygonal openings into which components, such as light fixtures, can be inserted and supported. Another proposal is to place the LV light system into an exposed ceiling system where units can be attached to a pendant which is attached to a structure. The UV lights may also be surface mounted to a ceiling or wall, integrated with drywall or even be portable.

Ultraviolet (UV) light has long been used as a disinfectant in wastewater treatment, hospital rooms, and other settings. In general, UV light is classified into four wavelength ranges: vacuum UV, from about 100 nanometers (nm) to amount 200 nm, UV-C, from about 200 nm to about 280 nm; UV-B, from about 280 nm to about 315 nm; and UV-A, from about 315 nm to about 400 nm. Generally, ultraviolet light, and in particular, UV-C light is “germicidal,” i.e., it deactivates the DNA of bacteria, viruses, fungi, and other pathogens and thus destroys their ability to multiply and cause disease. This effectively results in sterilization of the microorganisms.

Specifically, UV-C light causes damage to the nucleic acid in DNA by forming covalent bonds between certain adjacent bases in the DNA. The formation of these bonds prevents the DNA from being “unzipped” for replication, and the organism is neither able to produce molecules essential for life process, nor is it able to reproduce. In fact, when an organism is unable to produce these essential molecules or is unable to replicate, it dies. UV light with a wavelength of approximately between about 250 to about 280 nm provides the highest germicidal effectiveness. While susceptibility to UV light varies, exposure to UV energy of about 20 to about 34 milliwatt-seconds/cm² is adequate to deactivate approximately 99 percent of the pathogens.

The problem with incorporating UV-C lights into the modern environment is those lights also act on human DNA. This can lead to cancer. As a result, the lights must be segregated from the environment. However, this leads to the practical problem of subjecting the environmental air to the UV-C lights without subjecting the environments to the UV-C lights.

Consequently, there is a need for modular UV-C air circulation system that can be continuously run and optionally can be incorporated into a suspended ceiling, exposed system, surface mounted to a ceiling or wall, integrated with drywall or even be portable, so as to clean the air in the room(s).

### SUMMARY

Illustrative and alternative embodiments of an anti-microbial UVC based air purification system for a suspended ceiling are provided. The UV-C based anti-microbial air circulation system for a suspended ceiling, the air circulation system comprises a UV light assembly having an internal chamber containing at least one UVC light. The UV light assembly is connected to an intake assembly on one side and an exhaust assembly on the opposite side. Both the intake assembly and the exhaust assembly include an opening the side opposite the UV light assembly and the openings are in fluid communication with the internal chamber via an intake channel and an exhaust channel. As a result, environmental air may enter the intake opening flowing through the intake channel into the internal chamber then through to the exhaust channel and out the exhaust opening. The intake channel and the exhaust channel include a baffle assembly adapted to restrict UVC wavelength light from exiting from either the intake opening or the exhaust opening. Finally, a first fan is connected to the intake opening and a second fan connected to the exhaust opening. Both fans are positioned at an acute angle from vertical and are adapted to circulate air through the chamber wherein the air resides in the internal chamber for at least one tenth of one second (0.1 seconds).

In certain embodiments, the acute angle is from about 30 degrees to about 60 degrees. In such embodiments, the fan may be adapted to provide from about 40 cubic feet per minute (CFM) to about 150 CFM of airflow. In addition, to such airflow, the fan may be adapted to provide a pressure drop within the UV light assembly from about 0.25 inches of water to about 0.95 inches of water.

In certain embodiments, the UVC wavelength light is from about 250 nm to about 280 nm. In such embodiments, the environmental air may be subject to 15-65 mJ/cm² of 254 nm light.

In certain embodiments, the environmental air resides in the internal chamber from about 0.2 seconds to about 0.5 seconds.

In certain embodiments, the baffle assembly includes an upper baffle and a lower baffle wherein both the upper baffle and lower baffle take on a wave shape with the highest point of both baffles are closer to the openings than the lowest points of both baffles, which are closest to the UV light assembly.

In certain embodiments, the intake assembly further includes a filter.

In other embodiments, an outer shell including a pocket with a lockable door encloses the UV light assembly, intake assembly, and exhaust assembly. The shell provides protection to the assemblies to prevent the release of UVC wavelength light into the environment.

These and other features, aspects, and advantages of the invention will become better understood with reference to the following description and the appended claims.

### BRIEF DESCRIPTION OF THE FIGURES

Additional aspects, features, and advantages of the invention, as to its system, structure, components, configuration, and operability will be understood and become more readily apparent when the invention is considered in light of the following description of the figures made in conjunction with the accompanying drawings, wherein:

FIG. 1 shows an exemplary embodiment of the air circulatory system disclosed herein.

FIG. 2 shows an exemplary embodiment of the outer shell disclosed herein.

FIG. 3A shows a cross sectional view of the outer shell disclosed herein.

FIG. 3B shows a cross sectional view of the door hinge disclosed herein when closed.

FIG. 3C shows a cross sectional view of the door hinge disclosed herein when open.

FIG. 4 shows an exemplary embodiment of intake assembly disclosed herein.

FIG. 5 shows an exemplary embodiment of the exhaust assembly disclosed herein.

FIG. 6 shows an exemplary embodiment of the electronics assembly disclosed herein.

FIG. 7 shows an exemplary embodiment of the baffle assembly disclosed herein.

FIG. 8 shows an exemplary embodiment of the air circulatory system disclosed herein.

### DETAILED DESCRIPTION

The features and benefits of the disclosure are illustrated and described by reference to exemplary embodiments. The disclosure also includes the drawing. This description of exemplary embodiments is intended to be read in connection with the accompanying drawing, which is to be considered part of the entire written description. Accordingly, the disclosure expressly should not be limited to such exemplary embodiments illustrating some possible non-limiting combination of features that may exist alone or in other combinations of features.

In the description of embodiments, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top,” and “bottom” as well as derivatives thereof (e.g., “horizontally,” “downwardly,” “upwardly,” etc.) should be construed to refer to the orientation as then described or as shown in the figure under discussion. These relative terms are for convenience of description only and do not require that the apparatus be construed or operated in a particular orientation. Terms such as “attached,” “affixed,” “connected,” “coupled,” “interconnected,” and similar terms refer to a relationship wherein structures are secured or attached to one another either directly or indirectly through intervening structures, as well as both moveable or rigid attachments or relationships, unless expressly described otherwise.

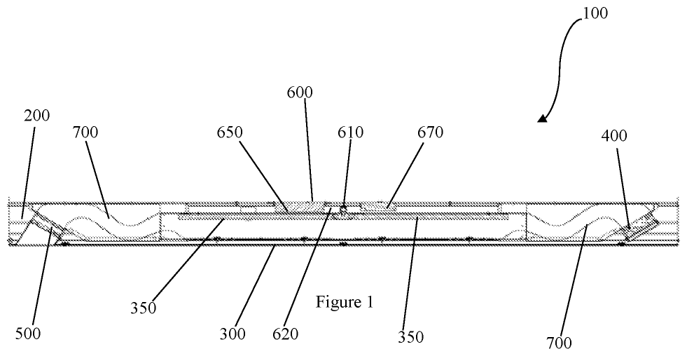

Referring to FIG. 1, the present air circulation system 100 for a suspended ceiling includes an outer shell 200 enclosing a UV light assembly 300 containing at least one UV-C light 350. The UV light assembly 300 is bookended by an intake assembly 400 one side and an exhaust assembly 500 on the other side. Both the intake assembly 400 and the exhaust assembly 500 are connected to the UV light assembly 300 with fasteners. An electronics assembly 600, having a ballast 650 and a power supply 670 connected to the bottom of the electronics assembly 600 with fasteners, is itself removably connected with fasteners to the tops of the outer shell 200 and the UV light assembly 300. To prevent light from escaping into the exterior environment, both the intake assembly 400 and the exhaust assembly 500 include a baffle assembly 700.

Outer Shell

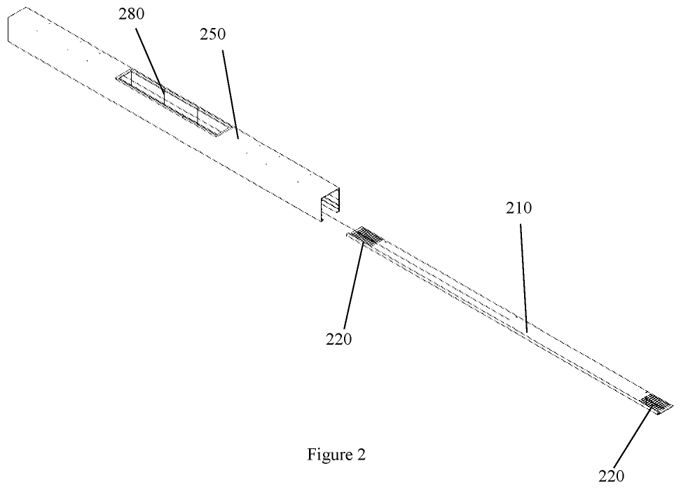

Referring to FIG. 2, the outer shell 200 comprises a door 210 removably connected to a pocket 250. The top portion 252 of the outer shell 200 also contains an electronics cutout 280 onto which the electronics assembly 600 may be placed so that the ballast 650 and power supply 670 are contained within the outer shell 200.

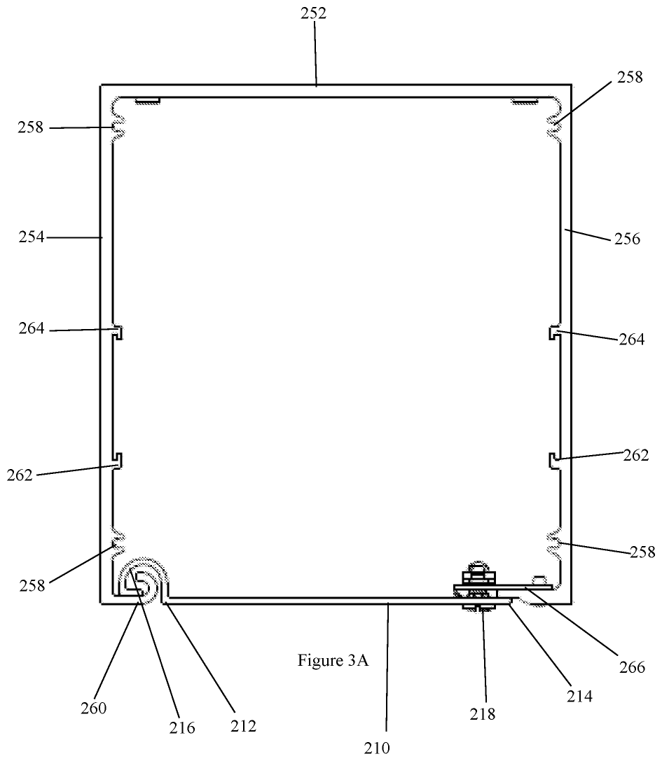

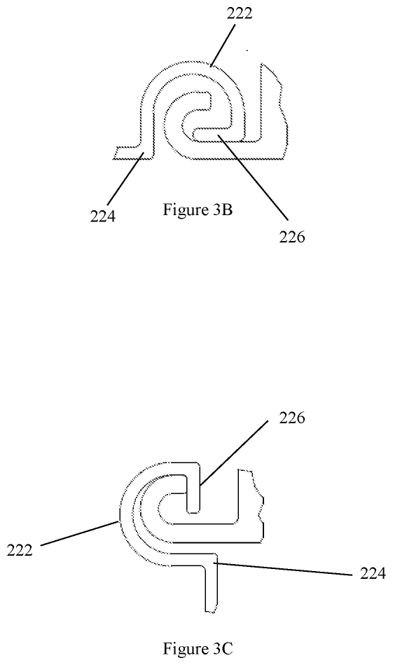

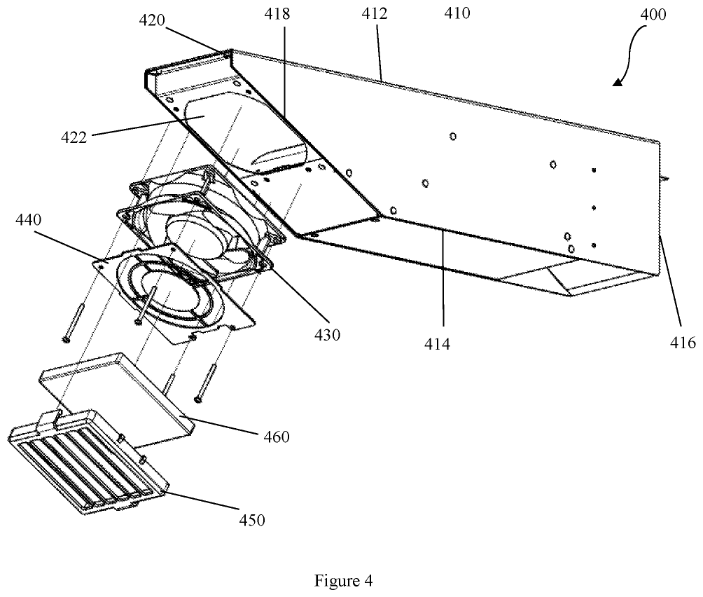

Referring to FIG. 3, the pocket includes a top portion 252 attached a first leg 254 and a second leg 256 on opposite ends of the top portion 252. The first leg 254 and the second leg 256 extend downward from the top portion 252 and are preferably, but not necessarily, substantially parallel to each other and substantially perpendicular to the top portion 252. The bottom of the pocket 250 is open. As used herein, the “interior” of the pocket 250 is defined as the region between the first leg 254 and the second leg 256, and the “exterior” of the pocket 250 is any space not between the first leg 254 and the second leg 256, for example the space above the top portion 252, the space to the left of the first leg 254, and the space to the right of the second leg 256. Accordingly, the interior surface of the pocket 250 is the surface adjacent to the interior of the pocket 250 and the exterior surface of the pocket 250 is the surface adjacent to the exterior of the pocket 250.

The interior of the pocket 250 includes: (1) a plurality of screw holes 258 for attaching an end cap or cover to the pocket 250, and (2) a door hook 260. The screw holes may be located on the top portion 252, the first leg 254, or the second leg 256. The door hook 260 projects outward from the interior surface of the first leg 254 towards the second leg 256 curving back around towards the top portion 252 till it points towards the first leg 254. While the door hook 260 projects outward from the terminal end of the first leg 254 in FIG. 3, it will be understood, that the door hook 260 may project outward from any location on the interior surface of the first leg 254 between the terminal end of the first leg 254 and the end of the first leg 254 that connects the first leg 254 to the top portion 252.

The interior of the pocket 250 may also include channels formed by a first hook 262 and an opposite facing hook 264. In certain embodiments, channels and the hooks forming them 262, 264 may be on any interior surface of the top portion 252, first leg 254, or second leg 256.

It will be understood that the pocket 250 may also include any number of additional protuberances on the interior or exterior of the pocket 250 which are not shown to attach additional components to the pocket 250. For example, a ceiling beam may attach to the second leg 256, and a hang wire may attach to the top portion 252 or the first leg 254.

The interior of the pocket 250 also includes a door projection 266 which projects outward from the second leg 256. In certain embodiments, the door projection 266 projects outward at a substantially perpendicular angle from the interior surface of the second leg 256. The door projection 266 includes at least one hole adapted to align with a hole in the door 210 permitting a door fastener 218 to pass through both holes and releasable close up the interior of the pocket 250 with the door 210.

The interior of the pocket 250 may also include one or more additional pluralities of protuberances (not shown), also commonly referred to as bosses, for attaching components to the interior of the pocket 250. For example, the plurality of protuberances which are not shown may be used to attach a wire management element to the pocket 250.

The door 210 is substantially planar with a first edge 212 and an opposite second edge 214. A pocket hook 216 adapted to mate with the door hook 260 projects outward from the plane of the door 210 at a location closer to the first edge 212 than the second edge 214. In certain embodiments, the pocket hook 216 includes a circular section 222 having a radius that is larger than the radius of the door hook 260. The circular section 222 is attached to the door 210 at an attachment point 224. An engagement flange 226 projects into the interior of the circular section 222. The engagement flange is adapted to engage with the pocket hook as shown in FIGS. 3B and 3C. In certain embodiments, the engagement flange 226 projects in a manner that is substantially parallel to the plane of the door 210. In certain embodiments, such as those depicted in FIGS. 3B and 3C, the engagement flange 226 does not reside within the same plane of the door 210. In other embodiments, the engagement flange may reside within the same plane as the door 210.

Although in FIG. 3 the pocket hook 216 projects outward from the first edge 212, it will be understood that the pocket hook 216 may project outward from the door 210 at any location closer to the first edge 212 than the second edge 216.

The door 210 also includes at least one hole adapted to align with a hole in the pocket 250 permitting a door fastener 218 to pass through both holes and releasable close up the interior of the pocket 250 with the door 210.

In certain embodiments the door may include a plurality of slats, slots, diverters, deflectors, and/or diffusers 220.

UV Light Assembly

Referring to FIG. 1, the UV light assembly 300 containing at least one UV-C light 350. The UV light assembly 300 includes a hollow channel in fluid communication with both the intake assembly 400 on one side and the exhaust assembly 500 on the other side. Thus, air may enter the intake assembly 400 pass through the UV light assembly 300 and exit out the exhaust assembly 200.

In certain embodiments the UV light assembly 300 will have a height of about 3.625 inches (9.2 cms), a width of about 4.75 inches (12.1 cms), and a length of about 44 inches (111.8 cms). About being plus or minus 0.05 inches (0.1 cms). Thus the cross sectional area (length×height) will be about 17.25 inches² (43.8 cm²).

The UV-C light 350 may emit any wavelength of UV-C light. The wavelength emitted from the UV-C light 350 may be from about 200 nm to about 280 nm or any combination thereof. About being plus or minus 1.5 nm. For example, it may be about 222 nm, about 254 nm, about 267 nm, about 280 nm, or any combination thereof. In certain embodiments, the UV-C light will emit about 254 nm wavelength light.

The UV light assembly 300 is adapted to subject air passing through to at least about 300 mJ/cm² of 254 nm light. About being plus or minus 2 mJ/cm². In some embodiments, the UV light assembly 300 is adapted to subject air passing through to at least about 250 mJ/cm² of 254 nm light. In some embodiments, the UV light assembly 300 is adapted to subject air passing through to at least about 200 mJ/cm² of 254 nm light. In some embodiments, the UV light assembly 300 is adapted to subject air passing through to at least about 150 mJ/cm² of 254 nm light. In some embodiments, the UV light assembly 300 is adapted to subject air passing through to at least about 100 mJ/cm² of 254 nm light. In some embodiments, the UV light assembly 300 is adapted to subject air passing through to at least about 50 mJ/cm² of 254 nm light. In some embodiments, the UV light assembly 300 is adapted to subject air passing through to at least about 25 mJ/cm² of 254 nm light. In some embodiments, the UV light assembly 300 is adapted to subject air passing through to at least about 15 mJ/cm² of 254 nm light.

In certain embodiments, the UV dosage provided by the UV-C light 350 may be from about 15 mJ/cm² to about 65 mJ/cm². About being plus or minus 0.5 mJ/cm². For example, the UV dosage provided by the UV-C light 350 may be about 16 mJ/cm² or about 21 mJ/cm². In other embodiments, the UV dosage provided by the UV-C light 350 may be from about 15 mJ/cm² to about 25 mJ/cm². In other embodiments, the UV dosage provided by the UV-C light 350 may be from about 16 mJ/cm² to about 21 mJ/cm². In other embodiments, the UV dosage provided by the UV-C light 350 may be about 16 mJ/cm².

In certain embodiments the residence time within the UV light assembly 300 may be from about 0.1 to about 0.55 seconds. About being plus or minus 0.02 seconds. In certain embodiments the residence time within the UV light assembly 300 may be from about 0.15 to about 0.5 seconds. In certain embodiments the residence time within the UV light assembly 300 may be from about 0.2 to about 0.5 seconds. In certain embodiments the residence time within the UV light assembly 300 may be from about 0.2 to about 0.35 seconds. For example, the residence time within the UV light assembly 300 may be about 0.23 seconds or about 0.3 seconds. In certain embodiments the residence time within the UV light assembly 300 may be from about 0.2 to about 0.3 seconds. In certain embodiments the residence time within the UV light assembly 300 may be from about 0.3 to about 0.35 seconds.

To obtain the described residence time in the UV light assembly 300, the speed of the fan within the intake assembly 400 or the exhaust assembly 500 may be adjusted.

Intake Assembly

Referring to FIG. 4, the intake assembly 400 includes an intake housing 410 and a fan 430 attached to the intake housing 410 with fasteners. The intake assembly may also include a fan guard plate 440, a filter basket 450 and optionally a filter 460. The intake assembly 400 include a channel with an opening 422 that is in fluid communication between the fan 430 on one side and the chamber of the UV light assembly 300 on the other side.

When viewed from the side, the intake housing 410 may take the shape of a rectangle, square, or other polygon. As depicted in FIG. 4, when viewed from the side, the intake housing 410 may include a first side 412 that is substantially parallel with a second side 414. The intake housing 410 may also include a third side 416 that is substantially perpendicular with one edge of first side 412 and the same edge on the second side 416. Furthermore, in certain embodiments, the first side 412 may be longer than the second side 414. An extension 420 projects substantially perpendicular out from the edge of first side 412 opposite of the edge where the third side 416 intersects the first side 412.

In certain embodiments, a fourth side 418 connects the second side 414 to the terminal edge of the extension 420. Thus, the fourth side 418 forms an obtuse angle with the terminal edge of the projection 420 and an obtuse angle with the second side 414. In certain embodiments, the obtuse angle formed by the fourth side 418 contacting the terminal edge of the extension 420 is from about 100 degrees to about 150 degrees. About being plus or minus 2 degrees. In certain embodiments, the obtuse angle formed by the fourth side 418 contacting the terminal edge of the extension 420 is from about 110 degrees to about 140 degrees. In certain embodiments, the obtuse angle formed by the fourth side 418 contacting the terminal edge of the extension 420 is from about 120 degrees to about 135 degrees. In certain embodiments, the obtuse angle formed by the fourth side 418 contacting the terminal edge of the extension 420 is about 120 degrees.

In certain embodiments, the fourth side 418 may also be substantially perpendicular to the first side 412. In other embodiments, the fourth side 418 may be substantially parallel to the first side 412.

In certain embodiments, the fan 430 is at substantially perpendicular angle to the first side 412. In other embodiments, the fan 430 is at an angle substantially parallel to the fourth side 418. For example, the fan may be at an angle that is from about 30 degrees to about 60 degrees from the longer first side 412. The fan 430 may be adjustable and adapted to provide from about 40 cubic feet per minute (CFM) to about 150 CFM of airflow. About being plus or minus 2 CFM.

In other embodiments, The fan 430 may be adjustable and adapted to provide from about 55 CFM to about 130 CFM of airflow. For example, the fan 430 may be adjustable and adapted to provide either 115 CFM or 90 CFM of airflow. In other embodiments, the fan 430 may be adapted to provide about 50 CFM or 100 CFM of airflow.

In certain embodiments, the fan 430 may cause a pressure drop within the UV light assembly 300 of between about 0.15 inches of water to about 1.5 inches of water. About being plus or minus 0.02 inches. For example the velocity pressure within the UV light assembly 300 may be about 0.153 inches of water at 50 CFM or about 0.585 inches of water at 100 CFM. In other embodiments, the fan 430 may cause a pressure drop within the UV light assembly 300 of between about 0.25 inches of water to about 0.9 inches of water. For example the velocity pressure within the UV light assembly 300 may be about 0.4 inches of water or about 0.8 inches of water.

In certain embodiments, the filter 460 may be an air filter, such as a MERV 4, 5, or 6 filter.

Exhaust Assembly

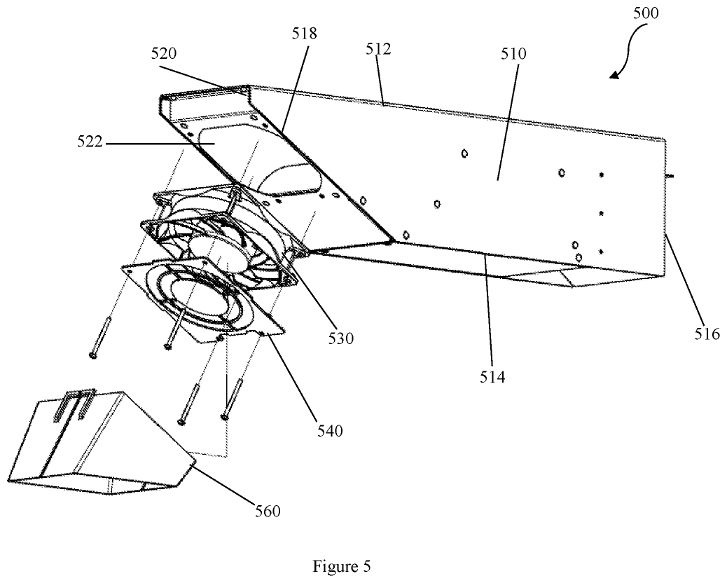

Referring to FIG. 5, the exhaust assembly 500 includes an exhaust housing 510 and a fan 530 attached to the exhaust housing 510 with fasteners. The exhaust assembly 500 may also include a fan guard plate 540 and an exhaust vent 560. The exhaust assembly 500 includes a channel with an opening that is in fluid communication between the chamber of the UV light assembly 300 on one side and the and the exhaust fan 530 on the other side.

When viewed from the side, the exhaust housing 510 may take the shape of a rectangle, square, or other polygon. As depicted in FIG. 5, when viewed from the side, the exhaust housing 510 may include a first side 512 that is substantially parallel with a second side 514. The exhaust housing 510 may also include a third side 516 that is substantially perpendicular with one edge of first side 512 and the same edge on the second side 516. Furthermore, in certain embodiments, the first side 512 may be longer than the second side 514. A flange 520 projects substantially perpendicular out from the edge of first side 512 opposite of the edge where the third side 516 intersects the first side 512.

In certain embodiments, a fourth side 518 connects the second side 514 to the terminal edge of the flange 520. Thus, the fourth side 518 forms an obtuse angle with the terminal edge of the flange 520 and an obtuse angle with the second side 514. In certain embodiments, the obtuse angle formed by the fourth side 518 contacting the terminal edge of the flange 520 is from about 100 degrees to about 150 degrees. About being plus or minus 2 degrees. In certain embodiments, the obtuse angle formed by the fourth side 518 contacting the terminal edge of the extension 520 is from about 110 degrees to about 140 degrees. In certain embodiments, the obtuse angle formed by the fourth side 518 contacting the terminal edge of the extension 520 is from about 120 degrees to about 135 degrees. In certain embodiments, the obtuse angle formed by the fourth side 518 contacting the terminal edge of the extension 520 is about 120 degrees.

In certain embodiments, the fourth side 518 may be substantially perpendicular to the first side 512. In other embodiments, the fourth side 518 may be substantially parallel to the first side 512.

In certain embodiments, the fan 530 is at substantially perpendicular angle to the first side 512. In other embodiments, the fan 530 is at an angle substantially parallel to the fourth side 518. For example, the fan may be at an angle that is from about 30 degrees to about 60 degrees from the longer first side 512. The fan 530 may be adjustable and adapted to provide from about 40 CFM to about 150 CFM of airflow. About being plus or minus 2 CFM. In other embodiments, The fan 530 may be adjustable and adapted to provide from about 55 CFM to about 130 CFM of airflow. For example, the fan 530 may be adjustable and adapted to provide either 115 CFM or 90 CFM of airflow. In other embodiments, the fan 530 may be adapted to provide about 50 CFM or about 100 CFM of airflow.

In certain embodiments, the fan 530 may cause a pressure drop within the UV light assembly 300 of between about 0.15 inches of water to about 1.5 inches of water. About being plus or minus 0.02 inches. For example the velocity pressure within the UV light assembly 300 may be about 0.153 inches of water at about 50 CFM or about 0.585 inches of water at about 100 CFM. In other embodiments, the fan 530 may cause a pressure drop within the UV light assembly 300 of between about 0.25 inches of water to about 0.9 inches of water. For example the velocity pressure within the UV light assembly 300 may be about 0.4 inches of water or about 0.8 inches of water.

In certain embodiments, fans 430 and 530 may be adapted to combine to produce the desired CFM and/or pressure drop in inches of water.

In certain embodiments, the exhaust vent may direct airflow outward from the exhaust assembly 500 at an angle substantially perpendicular to the fourth side 518.

Electronics Assembly

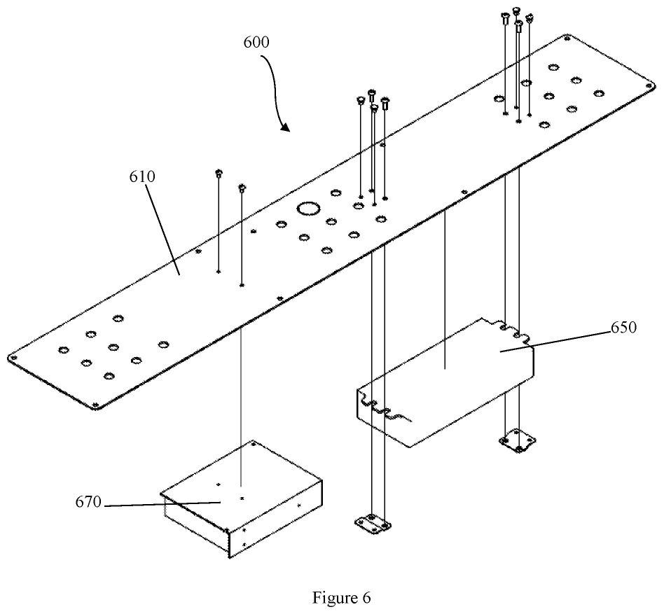

Referring to FIG. 6, the electronics assembly 600 has a top surface 610 and an opposite bottom surface 620. The top surface 610 is adapted to align with the exterior surface of the top portion 252 of the outer shell 200. Removably attached to the bottom surface 620 is a ballast 650 and a power supply 670. As outlined above, the electronics assembly 600 may be placed within the electronics cutout 280 of the outer shell 200 so that the ballast 650 and power supply 670 may be contained within the outer shell 200.

Baffle Assembly

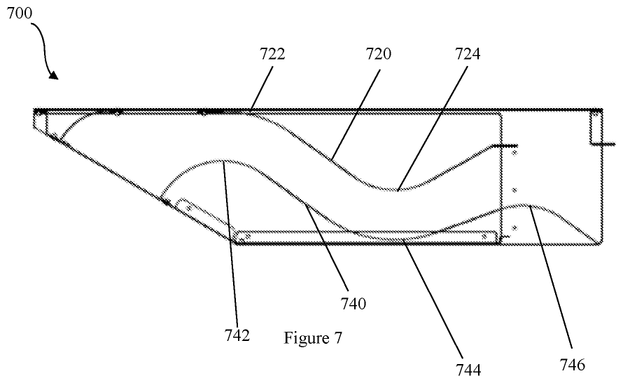

Referring to FIG. 7, the baffle assembly 700 is contained within both the intake housing 410 and the exhaust housing 510. The purpose of the baffle assembly 700 is to prevent UVC light from passing from the UV light assembly out through either of the intake opening 422 or the exhaust opening 522. The baffle assembly 700 includes an upper baffle 720 and a lower baffle 740. Both the upper baffle 720 and lower baffle 740 take on a wave shape. The upper crests 722 742 (i.e., the highest point on the baffles 720 740) are closer to the openings 422 522 than the troughs 724 744 (i.e., the lowest point on the baffles 720 740) which are closest to the UV light assembly 300. Furthermore, the trough 724 of the upper baffle 720 is lower than the crest 742 of the lower baffle 740. In addition, the lower baffle 740 includes a lower crest 746 which is further from the opening 422 522 than either of the upper crests 722 742 or either of the troughs 724 744.

The UV light has enough energy to reflect (bounce) off of the baffle surfaces multiple times before its energy is totally adsorbed. The required number of bounces is approximately 7 for full absorption. The lower crest 746 works in conjunction with the troughs 724 744 and the upper crests 722 742 as a light blocker to achieve this adsorption and thereby prevent the UV light from escaping into the exterior environment.

In this regard, the baffle assembly 700 prevents UVC light from passing from the UV light assembly out through either of the intake opening 422 or the exhaust opening 522 while still permitting environmental air to be circulated through the UV light assembly 300.

Multiple Filters

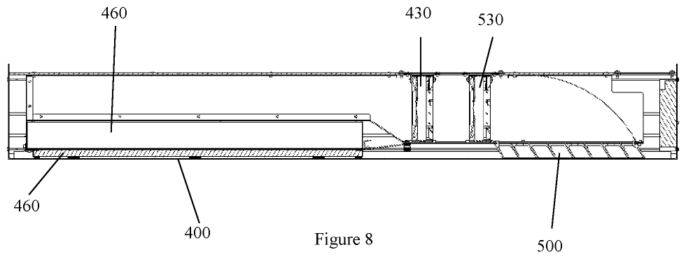

Referring to FIG. 8, the intake assembly 400 may be located on the bottom of the outer shell 200. Furthermore, two or more filters 460 may be aligned in series. For example, a MERV 4, 5, or 6 filter may be a first filter closer to the door 210 than a second filter, which may be a HEPA filter. The advantage of stacking the filters is the air is further purified before entering the hollow channel within the outer shell 200. As outlined above, the hollow channel is in fluid communication with both the intake assembly 400 on one side and the exhaust assembly 500 on the other side. In certain embodiments, only one intake fan 430 is required. In other embodiments, such as that depicted in FIG. 8, an intake fan 430 and second exhaust fan 530 are aligned in series. Such embodiments may optionally include a UV light assembly (not depicted).

Materials of Manufacture

It will be understood that the air circulation system 100, other than the electronic components (e.g., fans 430 530, ballast 650, power supply 670) or filter 460 may be constructed from any bendable material such as metals, polymers, or carbon fiber. In an exemplary embodiment, such non-electronic and filter components may be manufactured from extruded aluminum.

While this subject matter has been disclosed with reference to specific embodiments, it is apparent that other embodiments and variations can be devised by others skilled in the art without departing from the true spirit and scope of the subject matter described herein. The appended claims include all such embodiments and equivalent variations.

What is claimed is:

1. A UV-C based anti-microbial air circulation system for a suspended ceiling, the air circulation system comprising:
a UV light assembly having an internal chamber containing at least one UVC light;
the UV light assembly connected to an intake assembly on one side and an exhaust assembly on the opposite side, wherein both the intake assembly and the exhaust assembly include an opening the side opposite the UV light assembly and the openings are in fluid communication with the internal chamber via an intake channel and an exhaust channel so that environmental air may enter the intake opening flow through the intake channel into the internal chamber then through to the exhaust channel and out the exhaust opening;
the intake channel and the exhaust channel each include a baffle assembly adapted to restrict UVC wavelength light from exiting the air circulation system, and
a fan connected to either the intake opening or the exhaust opening, wherein the fan is adapted to circulate the environmental air through the chamber and wherein the environmental air resides in the internal chamber for at least about one tenth of one second.

2. The air circulation system of claim 1, wherein the fan is positioned at an acute angle from vertical.

3. The air circulation system of claim 2, wherein the acute angle is from about 30 degrees to about 60 degrees.

4. The air circulation system of claim 1, wherein the fan is adapted to provide from about 40 cubic feet per minute (CFM) to about 150 CFM of airflow.

5. The air circulation system of claim 1, wherein the fan is adapted to provide a pressure drop within the UV light assembly from about 0.25 inches of water to about 0.95 inches of water.

6. The air circulation system of claim 1, wherein the UVC wavelength light is from about 250 nm to about 280 nm.

7. The air circulation system of claim 1, wherein the environmental air is subject from about 15 mJ/cm² to about 65 mJ/cm² of 254 nm light.

8. The air circulation system of claim 1, wherein the environmental air resides in the internal chamber from about 0.2 seconds to about 0.5 seconds.

9. The air circulation system of claim 1, wherein the baffle assembly includes an upper baffle and a lower baffle wherein both the upper baffle and lower baffle take on a wave shape with the highest point of both baffles are closer to the openings than the lowest points of both baffles, which are closest to the UV light assembly.

10. The air circulation system of claim 1, wherein the intake assembly further comprises a filter.

11. A UV-C based anti-microbial air circulation system for a suspended ceiling, the air circulation system comprising:
a UV light assembly having an internal chamber containing at least one UVC light;
the UV light assembly connected to an intake assembly on one side and an exhaust assembly on the opposite side, wherein both the intake assembly and the exhaust assembly include an opening the side opposite the UV light assembly and the openings are in fluid communication with the internal chamber via an intake channel and an exhaust channel so that environmental air may enter the intake opening flow through the intake channel into the internal chamber then through to the exhaust channel and out the exhaust opening;
the intake channel and the exhaust channel each include a baffle assembly adapted to restrict UVC wavelength light from exiting the air circulation system;
a fan connected to either the intake opening or the exhaust opening, wherein the fan is adapted to circulate the environmental air through the chamber and wherein the environmental air resides in the internal chamber for at least about one tenth of one second; and
an outer shell comprising a door removably connected to a pocket, the pocket and door adapted to enclose the UV light assembly, intake assembly, and exhaust assembly.

12. The air circulation system of claim 11, wherein the fan is positioned at an acute angle from vertical.

13. The air circulation system of claim 12, wherein the acute angle is from about 30 degrees to about 60 degrees.

14. The air circulation system of claim 11, wherein the fan is adapted to provide from about 40 cubic feet per minute (CFM) to about 150 CFM of airflow.

15. The air circulation system of claim 11, wherein the fan is adapted to provide a pressure drop within the UV light assembly from about 0.25 inches of water to about 0.95 inches of water.

16. The air circulation system of claim 11, wherein the UVC wavelength light is from about 250 nm to about 280 nm.

17. The air circulation system of claim 11, wherein the environmental air is subject from about 15 mJ/cm² to about 65 mJ/cm² of 254 nm light.

18. The air circulation system of claim 11, wherein the environmental air resides in the internal chamber from about 0.2 seconds to about 0.5 seconds.

19. The air circulation system of claim 11, wherein the baffle assembly includes an upper baffle and a lower baffle wherein both the upper baffle and lower baffle take on a wave shape with the highest point of both baffles are closer to the openings than the lowest points of both baffles, which are closest to the UV light assembly.

20. The air circulation system of claim 11, wherein the intake assembly further comprises a filter.
